(19) United States
(12) Patent Application Publication
(10) Pub. No.: US 2011/0224761 A1
(43) Pub. Date: Sep. 15, 2011

(54) METHOD AND APPARATUS FOR COOLING BIOLOGICAL TISSUE

(75) Inventor: Dieter Manstein, Coral Gables, FL (US)

(73) Assignee: The General Hospital Corporation

(21) Appl. No.: 12/936,137

(22) PCT Filed: Apr. 1, 2009
(86) PCT No.: PCT/US09/39067

§ 371 Date: May 27, 2011

Related U.S. Application Data
(60) Provisional application No. 61/041,593, filed on Apr. 1, 2008.

(51) Int. Cl. A61F 7/00

(52) U.S. Cl. 607/105

(57) ABSTRACT

Exemplary embodiments of apparatus and method for tissue cooling using a plurality of hollow needles to deliver a cooled fluid into the tissue can be provided. For example, a volume and/or temperature of the fluid can be controlled to cool the tissue to a particular temperature or to within a predetermined temperature range. A tissue surface overlying the tissue to be cooled can be pre-cooled using conventional cooling techniques to increase the efficacy of the exemplary procedure. The exemplary method and apparatus can be used, e.g., to disrupt or damage fatty tissue that may then be reabsorbed by the body.

### CROSS REFERENCE TO RELATED APPLICATION

The present application claims priority from U.S. Provisional Patent Application Ser. No. 61/041,593 filed Apr. 1, 2008, the disclosure of which is incorporated herein by reference in its entirety.

### FIELD OF THE DISCLOSURE

The present disclosure is directed to a method and apparatus for cooling of tissue, and more particularly for targeted cooling of subdermal tissue by directing a cooled fluid into the tissue using an array of hollow needles.

### BACKGROUND INFORMATION

Cooling of biological tissue can be used for a variety of purposes. For example, cooling of the fatty tissue can disrupt such tissue, and can lead to a reduction in the amount of the fatty tissue present. Adipose or fatty tissue which includes lipid-rich cells can be selectively disrupted by cooling the tissue to a certain degree below normal body temperature. The disrupted fatty tissue may be resorbed by the body to some degree.

Excess fatty tissue can contribute to various health problems, such as hypertension, heart disease, osteoarthritis, and other conditions. The presence of the fatty tissue in various regions of the body may also be considered to be aesthetically undesirable. Reducing the amount of the fatty tissue present in various parts of the body, for both health and aesthetic reasons, is becoming more common. A variety of procedures, both invasive and non-invasive, can be used to remove the fatty tissue directly or to promote its resorption by the body.

Fatty tissue can include both subcutaneous fat and adipocytes (fat cells). Subcutaneous tissue can refer to tissue lying below the dermis. Various thicknesses of such fatty tissue may be present in different parts of the body. For example, large amounts of the fatty tissue are often found in the thighs, abdomen, and upper arms. In contrast, the facial region tends to have a thinner layer of the fatty tissue.

Liposuction is a conventional invasive procedure that can be used for removal of the fatty tissue from selected portions of a patient's body. Liposuction may be used, for example, to contour selected body parts such as the abdomen, buttocks, hips, thighs, etc. where larger deposits of the fatty tissue are present. Liposuction can also be referred to as suction lipectomy, lipolysis, or body contour surgery.

A conventional liposuction procedure can be performed, e.g., by inserting a hand-held tubular instrument (e.g., a cannula) through the patient's skin such that the tip of the cannula is within or adjacent to fat pockets in a target region of the tissue. The cannula can be moved around to mechanically disrupt and/or break up the fatty tissue, and pieces of the fatty tissue can then be aspirated through small openings along the sides or tip of the cannula using vacuum from a syringe or pump. The aspirated fatty tissue may then be deposited in a container provided in-line with the cannula and the vacuum source.

Conventional liposuction techniques and apparatus can produce undesirable side effects in a patient undergoing treatment. For example, neighboring tissue surrounding the fat being removed, such as blood vessels and connective tissue, may be significantly damaged or even partially removed along with the fatty tissue. Liposuction procedures can also lead to other adverse complications, including infection or excessive bleeding.

Disruption and/or removal of the fatty tissue can also be achieved by non-invasive techniques. Exercise can facilitate a reduction in the amount of the fatty tissue in the body. Certain nutritional supplements, when ingested, may also boost the body's metabolism and lead to an increased ‘burning’ of the fatty tissue. Certain compounds applied topically to the skin surface can be absorbed and may lead to a reduction in the amount of subcutaneous fatty tissue over time. However, such non-invasive techniques can have limited effectiveness and/or may require long times, e.g., on the order of weeks or months, to produce noticeable results. Targeting of specific regions of the fatty tissue may also not be easily achieved or even possible using such non-invasive techniques.

Other non-invasive techniques can be used for a reduction of the fatty tissue. For example, heating of such tissue can disrupt tissue structures and promote resorption of the fatty tissue by the body. Heating is generally performed by applying energy through the overlying skin to heat the fatty tissue. Forms of energy which can be used to thermally disrupt fatty tissue include ultrasound energy, which can be applied in the form of focused ultrasound waves. Electromagnetic energy, such as radiofrequency (RF) energy or laser energy, can also be used to heat the fatty tissue. Focusing of the applied energy below the skin surface and/or cooling of the skin surface can be performed to alleviate and/or prevent thermal damage to the dermis when the subcutaneous tissue is cooled. Because such energy applied from an external source passes through the dermis, localized heating of the fatty tissue and protection of dermal tissue from thermal damage can be difficult to achieve.

Cooling of subdermal tissue may be more difficult to target than heating of such tissue. For example, heating of the subdermal tissue (e.g., the fatty tissue) can be performed by focusing energy, e.g., ultrasound energy or electromagnetic energy, such that it is concentrated in a subdermal target region. A higher volumetric density of the energy can thereby be provided to the subdermal target region, where it can cause local heating by interacting with the tissue. Such application of focused (or unfocused) energy to the skin tissue can also be combined with superficial cooling before and/or during application of the energy. These procedures can produce an ‘inverted’ temperature profile in which the temperature of the deeper tissue is elevated above normal body temperature while the surface temperature can remain close to or even below normal body temperature. Such procedures can provide a heating of targeted regions of tissue below the surface while avoiding excessive heating and/or undesirable thermal damage of surface tissue such as the epidermis and dermis.

In contrast, cooling of a subdermal tissue may typically be performed by cooling a surface of the tissue such that tissue overlying the target region can be progressively cooled by a conduction. Conventional techniques that may be used for cooling tissue can include, for example, applying a cryospray or other chilled fluid or vapor to the surface of the skin, or contacting the surface of the skin with a cooled object. Cooling of deeper tissue can then occur by conduction of heat away from the deeper tissue through the overlying layers to the cooled surface. Such conduction can occur when the surface temperature is colder than the deeper tissue being cooled.

Cooling subdermal fatty tissue sufficiently to achieve a desired effect while avoiding damage or other undesirable cooling or freezing effects in the overlying tissue can be difficult to achieve. The extent of cooling of the fatty tissue can be limited by the amount of cooling that can be withstood by the overlying tissue. Certain techniques have been developed to more efficiently cool subdermal fatty tissue such as, e.g., “pinching” a portion of the tissue between one or more surrounding cold surfaces. This approach can increase the rate of cooling in the central region of the pinched portion of tissue by allowing heat to be extracted from a plurality of directions and by altering the geometry of the thermal conduction field. However, such techniques can still be limited by the amount of cooling that can be withstood by the surface tissue layers. Exemplary methods and apparatus for cooling fatty tissue, and certain effects of such cooling, are described in, e.g., U.S. Patent Publication No. 2003/0220674, and International Patent Publications WO 2007/127924 and WO 2007/133839.

Further, cooling of the deeper tissue can typically be based on a conduction of heat through overlying areas, and such diffusion may not be focused in a manner similar to that used for directing energy into a tissue for heating. Accordingly, it can be difficult to target particular areas of deeper tissue for cooling without affecting much of the adjacent and/or overlying tissue structures.

In view of the shortcomings of the above described procedures for tissue cooling and/or fat removal, it may be desirable to provide exemplary embodiments of methods and apparati that can combine safe and effective disruption of the selectively targeted fatty tissue by effectively cooling the fatty tissue while minimizing unwanted damage to surrounding tissue. Such exemplary procedures and apparati can reduce or minimize undesirable side effects such as intra-procedural discomfort, post-procedural discomfort, lengthy healing time, and/or damage of healthy tissue.

### SUMMARY OF EXEMPLARY EMBODIMENTS

Embodiments of the present disclosure are directed to methods and apparati for cooling tissue that can provide localized cooling of tissue below the tissue surface, while largely avoiding undesirable side effects, such as excessive cooling or freezing of the overlying tissue. For example, a cooled fluid can be dispersed directly within a target region of the tissue using, e.g., a plurality of hollow needles inserted into the skin. The hollow needles can be provided as a needle array that can be affixed to or coupled to a base or a substrate. The cooled fluid can include a saline solution, lidocaine, a detergent, an anti-inflammatory, and/or other components that can disrupt the targeted tissue and/or provide beneficial effects therein. The cooled fluid may also be liquid nitrogen.

A diameter of the hollow needles can be less than about 1000 μm, less than about 800 μm, less than about 500 μm, or even less than about 200 μm. A distance between the adjacent hollow needles can be less than about 10 mm, less than about 8 mm, or less than about 5 mm. A length of the needles protruding below a lower surface of the base can be at least about 1 mm, or between about 1 mm and about 5 mm, or greater than about 5 mm, depending on the location of the tissue region to be cooled. The needles can have the same length or some of the needles can have different lengths. An adjustable plate can be coupled to the base, such that the needles protrude through holes in the plate, and lengths of at least one of the needles protruding from the lower surface of the plate can be adjusted by adjusting the distance between the plate and the substrate. The array of needles can include, e.g., at least 10 needles, or at least 30 needles, or even at least 50 needles.

The cooled fluid can be provided from a reservoir, which can be an external reservoir or provided as part of the apparatus. The reservoir can be insulated and/or coupled to a cooling arrangement such as, e.g., a Peltier device or a conduit circulating a coolant. A switch can be provided to control a delivery of the cooled liquid into the tissue being cooled. The switch can be a mechanical switch configured to increase pressure within the reservoir, e.g., to force the cooled fluid from the reservoir into the hollow needles. Alternatively or in addition, the switch can be an electrical switch configured to activate an electric pump or a piezoelectric element to deliver the cooled fluid from the reservoir through the hollow needles and into the tissue.

The local temperature of the cooled tissue can be monitored, for example, using a temperature sensor provided in proximity to the tissue being cooled. Such temperature can be used to control an amount and/or temperature of the cooled fluid provided to the target region to achieve and/or maintain a particular temperature or temperature range of the cooled tissue. A display can be provided to indicate a temperature of the cooled fluid and/or the tissue being treated. Some or all of the components may be provided within a single housing, e.g., a handpiece or the like, and/or certain components may be provided outside of such housing (e.g., the reservoir).

A tissue surface overlying the tissue to be cooled may be pre-cooled using conventional techniques such as, e.g., a cryospray or a cold object placed in contact with the surface. Such pre-cooling can reduce or eliminate a pain sensation upon insertion of the hollow needles into the tissue, and can also increase the efficacy of the cooling procedure.

Exemplary embodiments of the present disclosure can provide, for example, a disruption and/or damage to the fatty tissue, which can promote a resorption of the cooled fatty tissue by the body.

These and other objects, features and advantages of the present disclosure will become apparent upon reading the following detailed description of exemplary embodiments of the present disclosure, when taken in conjunction with the appended claims.

### BRIEF DESCRIPTION OF THE DRAWINGS

Further objects, features and advantages of the present disclosure will become apparent from the following detailed description taken in conjunction with the accompanying figures showing illustrative embodiments, results and/or features of the exemplary embodiments of the present disclosure, in which:

FIG. 1 is a schematic illustration of an exemplary apparatus for targeted cooling of the fatty tissue according to exemplary embodiments of the present disclosure;

FIG. 2 is a schematic illustration of a distal portion of a hollow needle that can be used with the exemplary apparatus shown in FIG. 2, as well as with other exemplary embodiments of the apparatus as described and shown herein;

FIG. 3 is a schematic illustration of a further apparatus for disrupting the fatty tissue according to certain exemplary embodiments of the present disclosure;

FIG. 4A is a schematic view in plan of an exemplary array of hollow needles that can be used in certain exemplary embodiments of the present disclosure;

FIG. 4B is a schematic view in plan of another exemplary array of the hollow needles that can be used in further exemplary embodiments of the present disclosure; and

FIG. 5 is a schematic illustration of an exemplary apparatus that can be used to cool tissue according to yet further exemplary embodiments of the present disclosure.

Throughout the drawings, the same reference numerals and characters, unless otherwise stated, are used to denote like features, elements, components, or portions of the illustrated embodiments. Moreover, while the present disclosure will now be described in detail with reference to the figures, it is done so in connection with the illustrative embodiments and is not limited by the particular embodiments illustrated in the figures.

### DETAILED DESCRIPTION OF EXEMPLARY EMBODIMENTS

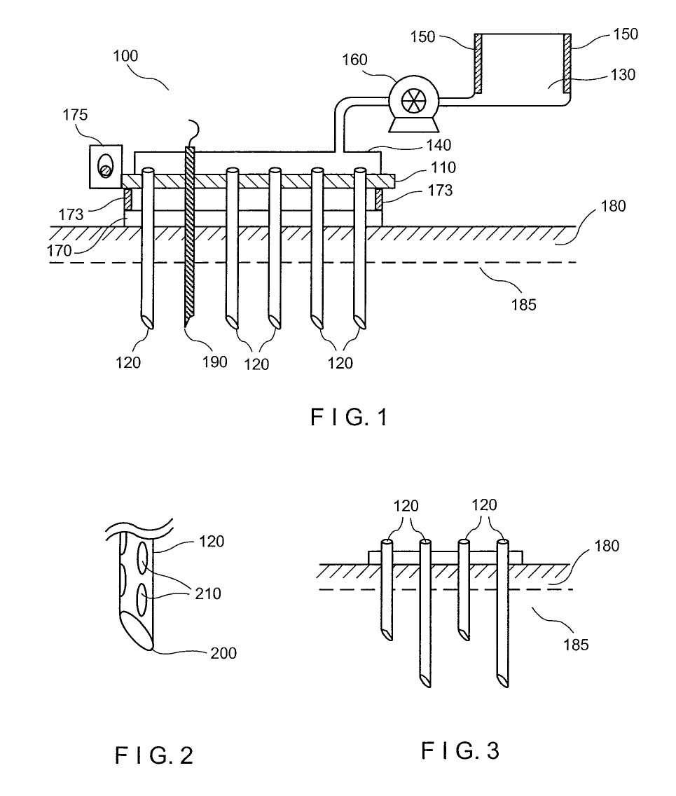

An exemplary embodiment of an apparatus 100 which can be used for targeted cooling of a tissue, e.g. a fatty tissue, according to certain exemplary embodiments of the present disclosure is shown in FIG. 1. The exemplary apparatus 100 can include a plurality of hollow needles 120 that can be affixed and/or mechanically coupled to a base 110 (which can be, e.g., a substrate, a portion of a housing, or the like). A conduit 140 can be provided in communication with a proximal end of the hollow needles 120, e.g., along or near an upper surface of the base 110. The conduit 140 can be provided in communication with a fluid reservoir 130. The fluid reservoir 130 can be configured or structured to hold a volume of a cooled fluid as described herein. For example, a cooling arrangement 150 can be provided in a thermal and/or fluid communication with the reservoir 130 and/or the reservoir 130 can be insulated. A pump 160 can be coupled to the conduit 140 to control an amount and/or rate of flow of the cooled liquid therethrough and into the hollow needles 120. An optional spacer plate 170 and/or posts 173 can be provided to adjust a distance at which the needles 120 extend from a lower surface of the spacer plate 170. A vibrating arrangement 175 can also be mechanically coupled to the base 110 and/or the needles 120 to facilitate insertion of the needles 120 into the tissue to be cooled, e.g., through the dermal layer 180 and into the subdermal fatty tissue 185. A temperature sensor 190 can also be provided to monitor the temperature of the cooled region of the fatty tissue 185.

Each hollow needle 120 can be formed or be composed of metal or another material that can be structurally rigid. The hollow needles 120 can have a cross-sectional shape that can be round, oval, or have any other shape that provides structural rigidity, and can include a central hollow core that facilitates a flow of a fluid therethrough.

The hollow needles 120 can have a sufficiently small diameter to facilitate their insertion into the tissue 180, 185 without causing significant tissue damage or pain, and they can also be large enough to facilitate the passage of fluid through the hollow central portion thereof. For example, the outside diameter of the needles 120 can be less than about 1000 μm, or less than about 800 μm. One or more of the hollow needles 120 having a diameter less than about 500 μm, for example, about 300 μm in diameter, can also be used if they are sufficiently stiff and/or strong for reliable insertion into and removal from skin or other tissue. One or more of the needles 120 that are wider than about 1000 μm in diameter can also be used in accordance with certain exemplary embodiments of the present disclosure; such larger needles can be more difficult to insert into the tissue, and can lead to an increased likelihood of pain and/or scarring as compared to the needles 120 that have a smaller diameter.

The inner diameter of each of the hollow needles 120 (e.g., the diameter of the central or interior hollow portion) can be sufficiently large to facilitate a flow of cooled liquid therethrough. For example, the inner diameter can be as large as possible while maintaining sufficient structural and mechanical strength of the needle 120 to facilitate insertion and removal thereof from the skin tissue 180, 185 without breaking. Alternatively, the needles 120 can have a somewhat smaller inner diameter and thicker material walls to provide an additional thermal insulation between the inner core of the needles 120 and the outer surface thereof.

The distal end 200 of each of the needles 120 can be cut, abraded, cast, molded, or otherwise shaped to form a sharp point or edge as shown in an exemplary embodiment thereof of FIG. 2. This sharp edge can facilitate a penetration of the distal end 200 of each of the hollow needles 120 into and through the dermal layer 180, such that it can be positioned proximal to or within the subcutaneous layer of the fatty tissue 185. The distal ends 200 can optionally be formed using a material that is different than a material used to form the walls of the needle 120 to further facilitate insertion of the needles 120 into the dermal layer 180 and/or the fatty tissue 185. For example, a portion of the needles 120 can be formed of and/or coated with a lubricant or low-friction material, such as Teflon®, to further facilitate a passage of the needles 120 through the dermal layer 180 and/or the fatty tissue 185.

In certain exemplary embodiments, one or more holes 210 can be provided through the walls of at least one of the hollow needles 120 near the distal end 200 thereof. These holes 210 can further facilitate flow of fluid through the central hollow portion of the needle 120 and into tissue surrounding the distal end 200.

The base 110 can be a plate, a portion of a housing, or another structure that can be configured or structured to hold the needles 120 in a particular configuration. For example, the base 110 can support the needles 120 such that at least some of the needles 120 are substantially parallel to each other to facilitate their insertion and removal from the tissue 180, 185. The base 110 can also be configured or structured to be attached and/or detached from a handpiece or other handle.

The length of the needles 120 protruding beyond a lower surface of the base 110 can be selected based on the depth of the fatty tissue layer 185 to be treated. For example, the depth of the fatty tissue 185 can be greater than about 3000 μm in many regions of the body. Accordingly, the lengths of the needles 120 can be greater than about 3000 μm for treating such regions. Shorter or longer needle lengths can also be used for treatment of different regions. For example, the lengths of the needles 120 can be shorter for treatment of facial regions, e.g., between about 1000 μm and about 3000 μm. Longer needle lengths, e.g., greater than about 3000 μm, can be provided for treatment of deeper regions of the fatty tissue 185 such as those located, e.g., in the upper arms, thighs, abdomen, etc.

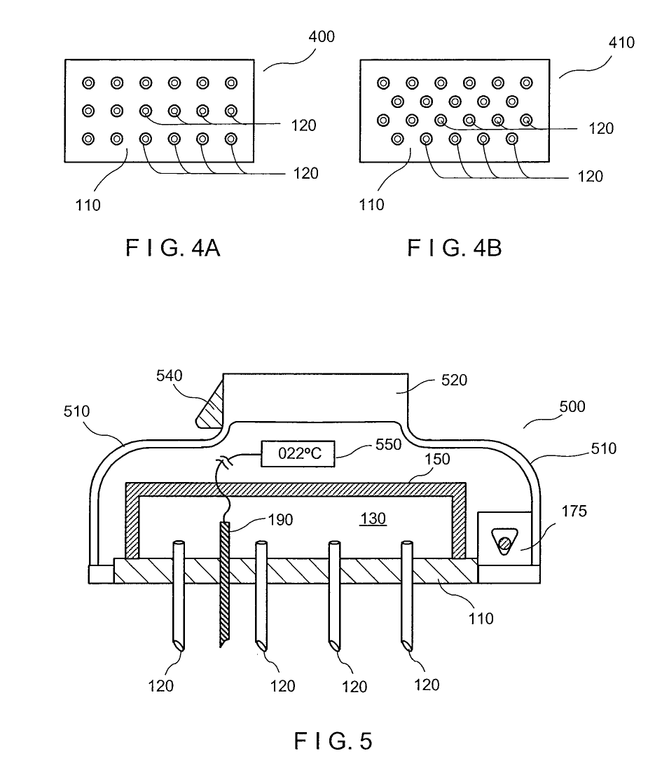

The hollow needles 120 can be provided with different lengths so that certain ones of the needles 120 extend to a plurality of different depths within the fatty tissue 185 when the array of the needles 120 is inserted into the dermal layer 180 and the fatty tissue 185, as shown in the exemplary embodiment of FIG. 4. This exemplary variation in needle lengths can facilitate treatment of a range of depths of the tissue, e.g., the fatty tissue 185, such that a larger volume of the tissue can be treated as described herein based on a single insertion of the hollow needles 120.

In certain exemplary embodiments, the apparatus 100 can include a spacer plate 170 provided between the base 110 and the surface of the tissue. The spacer plate 170 can be adjustably coupled to the base 110 by adjustment posts 173, which can be provided with a screw-type mechanism or other coupling arrangement that allows a distance between the spacer plate 170 and the substrate 110 to be controllably varied. The needles 120, which can be affixed to the base 110, can protrude through holes in the spacer plate 170. Accordingly, the length of the needles 120 that extends below the lower surface of the spacer plate 170 can be varied by adjusting the distance between the base and/or—substrate 110 and the spacer plate 170 to a particular value using the posts 173. The spacer plate 170 thus can facilitate selection and/or variation of the depth to which the needles 120 extend into the fatty tissue layer 185 when the needles 120 are inserted into the dermal layer 180 and the fatty tissue 185 until the spacer plate 170 contacts the surface of the tissue 180. The spacer plate 170 can also provide mechanical stability to the array of the hollow needles 120.

The lower surface of the base 110 and/or the spacer plate 170, if provided, can have a substantially planar shape or it can be contoured to follow a surface contour of the region of tissue being treated. For example, the bottom surface of the base 110 and/or the spacer plate 170 can be convex or concave, with any one of a range of curvatures.

According to certain exemplary embodiments of the present disclosure, the needles 120, the base 110 and/or the spacer plate 170, if provided, can be cooled prior to inserting the needles 120 into the tissue 180, 185. Cooling of at least certain portions of the exemplary embodiment of the apparatus 100 can be performed using any suitable technique (for example, by embedded conduits containing a circulating coolant, applying a cryospray, using a Peltier device, storing the apparatus in a cold enclosure, etc.) The cooled components of the apparatus 100 can assist in reducing or eliminating perceived pain when the needles 120 penetrate the dermal layer 180 and/or the fatty tissue 185.

The surface dermal region 180 can also be pre-cooled prior to the insertion of the needles 120, e.g., using any of a variety of conventional cooling techniques. For example, convective or conductive techniques, such as applying a cryospray or contacting the tissue surface with a cooled object (e.g., a cold piece of metal, a piece of ice, or the like) can be used. Such surface cooling can help to reduce and/or eliminate a perception of pain when the needles 120 are inserted. Pre-cooling of the dermal layer 180 and/or the fatty tissue 185 to be treated can also increase the efficacy of the exemplary cooling apparatus 100 described herein. For example, pre-cooling of the target tissue can require less heat to be extracted using the exemplary embodiments of the present disclosure described herein to achieve a particular level of cooling. Pre-cooling of tissue proximal to the target tissue can also reduce a flow of blood to the target region, which can reduce the rate of heating of the region by the blood. Accordingly, the pre-cooled tissue may be cooled more effectively.

In a further exemplary embodiment, a vibrating arrangement 175 can be mechanically coupled to the substrate 110 and/or the needles 120. The vibrating arrangement 175 can include, for example, a piezoelectric transducer, or a small motor with an eccentric weight fixed to the shaft thereof. Vibrations induced in the needles 120 by the vibrating arrangement 175 can facilitate a piercing of the surface of the dermal layer 180 by the needle tips and subsequent insertion of the needles 120 into the dermal layer 180 and/or the fatty tissue 185.

The vibrating arrangement 175 can provide an amplitude of vibration that can be between, e.g., about 50 μm and about 500 μm, or between about 100 μm and about 200 μm. The frequency of the induced vibrations can be between about 10 Hz and about 10 kHz, or between about 500 Hz and about 2 kHz, or about 1 kHz. Particular vibration parameters can be selected based, e.g., on the size, average spacing, and material of the needles 120, the number of needles in the apparatus 100, and physical characteristics of the tissue being treated. The vibrating arrangement 175 can optionally include a control arrangement configured to adjust the amplitude and/or frequency of the vibrations.

The needles 120 shown in the schematic side view of the exemplary apparatus 100 in FIG. 1 can be provided in a two-dimensional arrangement, where the needles 120 can be substantially parallel to each other, and/or can be oriented substantially perpendicular to the surface of the dermal layer 180 and/or the fatty tissue 185 being treated. The needles 120 can be arranged through the base and/or the substrate 110, for example, in a regular or near-regular square or rectangular pattern such as the exemplary pattern 400 as shown in FIG. 4A. The needles 120 can also be provided in the exemplary triangular pattern 410 as shown in FIG. 4B. Other exemplary patterns or arrangements of the needles 120 can be used, including non-uniform or irregular patterns.

The relative positions and spacing of the needles 120 can be selected based on a particular treatment to be performed. A spacing (e.g., lateral distance along the substrate 110) between adjacent needles 120 can be less than about 20 mm, or less than about 10 mm. Optionally, the spacing between the adjacent needles 120 in the array can be less than about 5 mm. Larger spacings, e.g., distances between at least some of adjacent one of the needles 120 that are about 30 mm or more, can also be used when selectively cooling larger volumes of the fatty tissue 185. The spacing between the needles 120 can not be uniform. For example, such exemplary spacing can be smaller in areas where a relatively greater amount of fat disruption or removal is desired.

Various numbers of the hollow needles 120 can be provided in the apparatus 100. For example, in certain exemplary embodiments, the apparatus 120 can include at least about 10 needles 120, at least about 30 needles 120, or at least about 50 needles 120. Arrays having a larger number of the needles 120 can be used, e.g., to cool a larger volume of tissue with a single insertion of the needle array into the skin. A larger region of the fatty tissue 185 can also be cooled by sequential insertion of the needles 120 in different locations along the skin surface. The number of the needles 120 provided in the exemplary apparatus 100 can be selected, e.g., based on various factors such as ease of manufacture, a particular needle spacing, a size of the region to be cooled, etc.

The exemplary apparatus 100 can facilitate a controllable delivery of the cold fluid from the reservoir 130 via the conduit 140 through the hollow needles 120, e.g., using the pump 160 or another device that can induce a flow of the cold fluid through the hollow needles 120 and into a portion of the tissue 185. The fluid can be precisely directed into predetermined regions of the fatty tissue 185 near the tips or distal ends of the needles 120. Accordingly, specific target regions of the fatty tissue 185 can be cooled directly by the cold fluid, while undesirable cooling or freezing of the dermal layer 180 located above the targeted fatty tissue 185 can be reduced and/or avoided. By using an array of such hollow needles 120, a large region of the fatty tissue 185 can be cooled by the cold fluid following a single insertion of the array of the hollow needles 120 into the tissue 180, 185.

In exemplary embodiments of the present disclosure, at least some of the portions of the fatty tissue 185 can be cooled to a temperature that is low enough to disrupt the fatty tissue structure without causing excessive necrosis. Such exemplary cooling can facilitate disruption of the cooled fatty tissue 185. For example, a target temperature of the cooled fatty tissue can be less than about 20° C., or about 0° C. Prolonged cooling of the fatty tissue 185 to temperatures significantly below about 0° C. can lead to freezing and possibly undesirable necrosis, fibrosis, and/or formation of scar tissue. However, cooling to such low temperatures can be performed in certain treatments.

The temperature of the cold fluid in the reservoir 130 can be controlled and/or maintained, e.g., by the cooling arrangement 150, which may include a control arrangement that allows the temperature to be set and maintained at a particular value. The reservoir 130 and/or conduit 140 can be insulated to provide better control of the fluid temperature when the cold fluid is provided to the hollow needles 120. In certain exemplary embodiments, the reservoir 130 can be provided or situated proximal to the conduit 140 and/or needles 120 to reduce heat losses from the cold fluid as it is provided to the needles 120. Such exemplary arrangement can also provide a more compact apparatus. In certain exemplary embodiments, the cooling arrangement 150 can include a Peltier device or the like, and can further include a control arrangement configured to maintain fluid within the reservoir 130 at a particular temperature that may be selectable.

The amount and/or flow rate of cold fluid delivered to a region of the fatty tissue 185 through the hollow needles 120 can be controlled, e.g., by the pump 160, which can further include a valve arrangement or the like. The amount and temperature of the cold fluid provided to cool the fatty tissue 185 can be controlled, e.g., based on a desired temperature to be achieved within the tissue 185 and/or a duration of cooling. In general, a colder fluid temperature and larger amounts of the cold fluid delivered to the target region can lead to a lower temperature of the targeted fatty tissue 185. The cooled tissue can gradually warm up as heat is transferred from surrounding warmer tissue to the cooled region.

The apparatus 100 can also include one or more temperature sensors 190. The temperature sensor 190 may be provided in a needle shape, and can have a sensing portion located near or at the distal end thereof that can be proximate to the distal end of one or more hollow needles 120. The sensor 190 can thus be configured to detect a local temperature within the target region of the fatty tissue 185 being treated, and optionally communicate with a display to show the local temperature in the fatty tissue 185. The sensor 190 can be configured to provide signals to control circuitry associated with the cooling arrangement 150 and/or the pump 160, e.g., in a feedback arrangement to more accurately achieve and/or maintain a particular temperature within the fatty tissue 185 by varying the temperature, flow rate, and/or amount of the cold fluid provided through the needles 120 into the fatty tissue 185.

The cold fluid provided to the targeted fatty tissue 185 can be water-based, for example, a saline solution or other fluid which can be introduced into and/or absorbed by the body without inducing significant undesirable side effects. The fluid can include one or more of an analgesic such as lidocaine, a detergent composition, an anti-inflammatory substance, a dispersing or emulsifying agent, etc. Various fluid components which can be used to disrupt or damage fatty tissue are described, for example, in U.S. Patent Publication No. 2006/0154906. Such components can also be used in exemplary embodiments of the present disclosure. The components of the cold fluid can be selected to enhance disruption and/or breakdown of the fatty tissue 185, e.g., when the tissue 185 warms up to normal body temperature after the cooling treatment has ended. The effectiveness of such components may be enhanced by the disruption of the structure of the fatty tissue 185 that can result from the cooling procedure described herein. Application of the cold fluids using the exemplary apparatus 100 described herein can also improve the efficacy of such fluid components by providing them in a well-dispersed state throughout the target fatty tissue 185 using the array of needles 120.

In certain exemplary embodiments, small amounts of a cryogenic fluid such as, e.g., liquid nitrogen can be introduced through the needles 120 and into the fatty tissue 185. The extremely cold temperature of liquid nitrogen can generate local tissue necrosis. The liquid nitrogen can be provided in quantities small enough such that it can be readily absorbed by the body when it vaporizes upon contact with the warm tissue 185.

In still further exemplary embodiments, the needles 120 can be formed using a material having a low thermal conductivity, such as a polymer, a ceramic, or the like. The distal ends of the needles can be formed using a more conductive material, such as a metal or alloy. Cold liquid provided into the needles 120 can further cool the distal ends of such needles, which can provide further cooling of the tissue 185 proximate to the distal portions of the needles 120.

A further exemplary apparatus 500 in accordance with yet additional exemplary embodiments of the present disclosure is shown in FIG. 5. The exemplary apparatus 500 can include the hollow needles 120 as described herein, that may be coupled to the base 110. The base 110 can be removably attached to a housing 510, and/or it can be formed as part of the housing 510. The (e.g., removable) base 110 can facilitate a replacement of the needles 120, such that different needle arrays may be used on different patients with a single apparatus 500. The housing 510 can also be provided, e.g., with a handle 520 or the like to facilitate positioning of the apparatus 500 and insertion of the needles 120 into the tissue to be cooled.

An exemplary embodiment of the fluid reservoir 130 configured or structured to hold a volume of a cooled fluid as described herein can be provided within the housing 510. The reservoir 130 can be in communication with a proximal end of the hollow needles 120, e.g., adjacent to an upper surface of the base 110. In certain exemplary embodiments, a chamber similar to the conduit 140 shown in FIG. 1 can be provided in communication with the fluid reservoir 130 and the proximal portions of the needles. Such exemplary chamber can facilitate a flow of the cold fluid from the reservoir 130 into and through the hollow needles 120, and then into, e.g., the tissue proximal to the distal ends of the needles 120. The reservoir 130 can be insulated and/or a cooling arrangement 150, e.g., a Peltier device or the like, can be provided in thermal communication with the reservoir 130.

A switch 540 can be provided in the housing 510 to control an amount and/or rate of flow of the cooled liquid from the reservoir 130 into the hollow needles 120. For example, the switch 540 can be a trigger that is configured to increase pressure within the reservoir 130 when activated, which can force at least some of the cooled fluid through the needles 120 and into the tissue being cooled. Alternatively or in addition, a pump arrangement similar to the pump 160 shown in FIG. 1 can be provided, and the switch 540 can be configured to activate the pump arrangement to force fluid into the needles 120. For example, such exemplary pump arrangement can include a piezoelectric element that is configured to deform when activated and propel at least some of the cooled fluid from the reservoir 130 into the needles 120.

A spacing arrangement similar to the spacer plate 170 and the posts 173 shown in FIG. 1 can also be provided in the exemplary apparatus 500 to adjust a length of the needles 120 protruding from a lower surface of the apparatus 500. The vibrating arrangement 175 can also be coupled to the housing 510, the base 110, and/or the needles 120 to facilitate the insertion of the needles 120 into the tissue.

The temperature sensor 190 can also be provided to monitor the temperature of the tissue being cooled, as described herein. The apparatus 500 can also include, e.g., a display arrangement 550 that can be configured to display the fluid temperature in the reservoir, the target tissue temperature, etc. For example, the switch 540 can be manually operated at appropriate intervals to attain and/or maintain a desired temperature of the target tissue, where such temperature can be shown on the display arrangement 550. The apparatus 500 can also include a power source provided therein that is configured to activate any components that may utilize of require such power (e.g., temperature sensor circuitry, the display arrangement 550, the pump arrangement, etc.). Alternatively or in addition, power can be provided to the apparatus 500 from an external power source, e.g., from an electrical outlet via an electrical cord or the like.

According to another exemplary embodiment of the present disclosure, a method can be provided for cooling the fatty tissue 185, which can be located beneath the dermis layer 180. Such exemplary fatty tissue 185 can be cooled to a preselected temperature for a particular duration by introducing a cold fluid into the tissue. For example, the cold fluid can be directed into the fatty tissue 185 using the needles 120, as shown in FIG. 1. The cold fluid can be provided, e.g., from the reservoir 150 that can be configured to adjust or maintain a temperature of the liquid to a predetermined value. Low temperatures and/or components in the fluid can disrupt or damage the cooled fatty tissue 185, and/or promote its resorption by the body. The array of the needles 120 can facilitate cooling of particular regions of the fatty tissue 185 by the application of the cold fluid directly to the fatty tissue 185. For example, controlled cooling of the deeper fatty tissue 185 may be achieved without requiring significant cooling of the skin overlying the target region.

A temperature of the target region of the fatty tissue 185 can be detected during the exemplary cooling procedure using, e.g., the temperature sensor 190 shown in FIG. 1. The flow rate and/or temperature of the cold fluid can also be controlled, e.g., based on a signal provided by the sensor 190 to achieve and/or maintain a particular temperature or range of temperatures within the targeted region of the fatty tissue 185. For example, the fatty tissue 185 can be cooled to a temperature of less than about 20° C., or to a temperature of about 0° C. Higher, or to lower temperatures. The fatty tissue 185 can also be maintained at one or more cold temperatures over a particular time interval by application of further volumes of the cold fluid through the needles 120 at certain times. Various combinations of cooling time and temperature can be provided by the exemplary embodiments of the method and apparatus described herein. Particular values for cooling time and temperature can be selected based on the particular tissue being treated and a desired effect.

The exemplary cooling techniques described herein can be performed in a single treatment, or by multiple treatments performed either consecutively during one session (e.g., one insertion of the array of needles 120 into a particular location of the dermal layer 180 and/or the fatty tissue 185). Such exemplary cooling can also be performed over longer time intervals using periodic applications of cold fluid into the fatty tissue 185. Multiple treatments can also be performed, e.g., at a single target region using multiple insertions of the needle arrays 120 described herein. The exemplary cooling can also be achieved over a larger area by inserting the needles 120 into a plurality of areas of the skin, and performing the tissue cooling techniques described herein in each area. Individual or multiple treatments of a given one or more regions of the tissue can be used to achieve the appropriate tissue damage and desired cosmetic effects.

The foregoing merely illustrates the principles of the present disclosure. Various modifications and alterations to the described embodiments will be apparent to those skilled in the art in view of the teachings herein. It will thus be appreciated that those skilled in the art will be able to devise numerous techniques which, although not explicitly described herein, embody the principles of the present disclosure and are thus within the spirit and scope of the present disclosure. All patents and publications cited herein are incorporated herein by reference in their entireties.

1-43. (canceled)

44. An apparatus for cooling a biological tissue, comprising:
a base; and
a plurality of hollow needles coupled to the base,
wherein the hollow needles are structured to be inserted simultaneously to one or more predetermined depths within the tissue;
wherein the hollow needles are structured to direct a cooled fluid into at least one portion of the tissue; and
wherein at least one of the hollow needles comprises a thermally insulating material.

45. The apparatus of claim 44, wherein the hollow needles comprise at least one of a polymer, a ceramic, or Teflon®.

46. The apparatus of claim 44, wherein at least one of the hollow needles includes a plurality of openings proximal to a distal end thereof.

47. The apparatus of claim 44, wherein at least two of the particular needles have a different length from one another.

48. The apparatus of claim 44, wherein a length of at least one of the hollow needles extending from a lower surface of the base is between about 1 mm and about 5 mm.

49. The apparatus of claim 44, wherein a length of at least one of the hollow needles extending from a lower surface of the base is greater than about 5 mm.

50. The apparatus of claim 44, wherein a distance between adjacent ones of the hollow needles is less than about 10 mm.

51. The apparatus of claim 44, wherein a distance between adjacent ones of the hollow needles is less than about 5 mm.

52. The apparatus of claim 44, wherein the hollow needles include at least 10 hollow needles.

53. The apparatus of claim 44, further comprising a vibrating arrangement that is mechanically coupled to the base.

54. The apparatus of claim 44, further comprising a movable plate provided in a proximity to the base such that the hollow needles pass through the movable plate, wherein a distance between the movable plate and the base is modifiable such that a length of the hollow needles extending from a lower surface of the movable plate is adjustable to a particular length.

55. The apparatus of claim 44, further comprising a conduit provided in communication with a proximal portion of the hollow needles, wherein the conduit is structured to direct the cooled fluid into the plurality of hollow needles.

56. The apparatus of claim 44, further comprising a temperature sensing arrangement, wherein at least a portion of the temperature sensing arrangement is provided in a location proximal to a distal portion of at least one of the hollow needles.

57. The apparatus of claim 44, further comprising a reservoir structured to hold the cooled fluid, wherein the reservoir is provided in communication with a proximal portion of at least one of the hollow needles.

58. The apparatus of claim 57, further comprising a temperature control arrangement configured to at least one of control or maintain a particular temperature of the cooled fluid within the reservoir.

59. The apparatus of claim 58, wherein the particular temperature is less than about 20° C.

60. The apparatus of claim 57, further comprising a display arrangement configured to display at least one of a temperature of the cooled liquid in the reservoir and/or a temperature of the tissue being cooled.

61. The apparatus of claim 44, wherein a diameter of at least one of the hollow needles is less than about 1000 μm.

62. The apparatus of claim 44, wherein a diameter of at least one of the hollow needles is less than about 500 μm.

63. A method for cooling a biological tissue, the method comprising:
simultaneously inserting a plurality of hollow needles into the tissue to position portions of the hollow needles at least one of within and/or proximal to at least a portion of the tissue; and
directing a cooled fluid through the hollow needles such that at least a portion of the heated fluid enters the tissue through at least one opening provided at the portions of the hollow needles,
wherein at least one of the hollow needles comprises a thermally insulating material; and
wherein at least one of a temperature or a volume of the cooled fluid directed into the tissue is selected to cool at least a portion of the tissue to a temperature within a predetermined temperature range.
